(19) United States
(12) Patent Application Publication
(10) Pub. No.: US 2012/0029333 A1
(43) Pub. Date: Feb. 2, 2012

(54) MODULAR INFUSION SET WITH AN INTEGRATED ELECTRICALLY POWERED FUNCTIONAL COMPONENT

(75) Inventors: Joerg Dogwiler, Bergdietikon (CH); Andreas Geipel, Oftringen (CH); David Teutsch, Schuepfen (CH); Christoph Huwiler, Baar (CH)

(73) Assignee: ROCHE DIAGNOSTICS INTERNATIONAL AG, Steinhausen (CH)

(21) Appl. No.: 13/015,063

(22) Filed: Jan. 27, 2011

(30) Foreign Application Priority Data
Jan. 28, 2010 (EP) 10152026

(51) Int. Cl. A61M 5/158; A61B 5/145; A61M 5/44

(52) U.S. Cl. 600/365; 604/151; 604/113

(57) ABSTRACT

An infusion set for administering a medicament delivered by an infusion pump which can be carried separately from the infusion set as well as an infusion system including the infusion set are disclosed. The infusion set can comprise a disposable part, a reusable part, and an electrically powered functional component. The disposable part can comprise a single lumen infusion cannula that projects from the underside and is the only skin piercing or penetrating element of the infusion set. The reusable part when in the interconnected state is fluidically isolated from the disposable part. The disposable part can comprise a feeding line which fluidically connects an upstream end of the first connector with a downstream end of a cannula to feed and deliver the medicament via the disposable part and bypass the reusable part.

### CROSS REFERENCE TO RELATED APPLICATION

This application claims the priority to European Application No. EP10152026 filed Jan. 28, 2010, which is hereby incorporated by reference in its entirety.

### TECHNICAL FIELD

The disclosure relates to an infusion set for attachment at an infusion site on a person's skin to administer a medicament delivered by a remote infusion pump. The disclosure is also related to an infusion system comprising the infusion pump and the infusion set and furthermore to an infusion and monitoring system comprising the infusion system and a monitoring system for monitoring a parameter characteristic for the health condition of the person and relevant for the administration of the medicament. The disclosure is concerned in particular with diabetic therapy, i.e. the administration of insulin, but is advantageous also for other therapies and the administration of other infusable medicaments.

### BACKGROUND

Infusion systems that deliver medication by infusion are typically divided into an infusion pump which can be carried on or under the clothing and an infusion set which can be attached at an infusion site directly on a person's skin such that a stiff or soft cannula projecting from an adhesive underside of the infusion set is placed with its tip in body tissue. Known infusion sets can conveniently be worn directly on the skin while the infusion pump including a medicament reservoir, feeding means, an energy source, control unit, manipulator, alarm means and the like is worn remote from the infusion set. The small and light-weight infusion sets are replaced in short intervals for reasons of sterility, in diabetic therapy typically every two to three days, and must be inexpensive therefore.

The functionality of infusion systems, not the least those for self-administration of the respective medicament, are ever increasing, and so do the safety requirements, despite the demand for keeping the associated costs low. Infusion systems may be coupled with monitoring systems for monitoring a biological parameter on which the dosed administration depends.

### SUMMARY

Included are embodiments of an infusion set for administering a medicament delivered by an infusion pump which can be carried separately from the infusion set. The infusion set can comprise a disposable part, a reusable part, and an electrically powered functional component. The disposable part can comprise an adhesive underside for attachment at an infusion site on a person's skin, a single lumen infusion cannula which projects from the underside and is the only skin piercing or penetrating element of the infusion set, a first connector which fluidically connects the cannula to the infusion pump, and a second connector. The reusable part can comprise an energy source which electrically powers the functional component, and a third connector mated with the second connector of the disposable part to interconnect the disposable part and the reusable part. The reusable part is in the interconnected state fluidically isolated from the disposable part. The disposable part can comprise a feeding line fluidically connecting an upstream end of the first connector with a downstream end of the cannula to feed and deliver the medicament via the disposable part and bypass the reusable part.

In another embodiment, an infusion system can comprise an infusion set, an infusion pump with a medicament reservoir, feeding means for feeding medicament from the reservoir, an energy source for powering the feeding means, and flexible tubing connecting the infusion pump with the infusion set to administer the medicament from the reservoir via the cannula of the infusion set, the tubing being directly connected releasably or unreleasably with the disposable part.

In a further embodiment, the infusion system can comprise a monitoring system which includes a sensor which senses a therapy relevant health parameter of the person, the sensor being separate from the infusion set.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 depicts an infusion set with a disposable and a reusable part in an interconnected state according to one or more embodiments shown and described herein;

FIG. 2 depicts an infusion set with the reusable part disconnected from the disposable part according to one or more embodiments shown and described herein;

FIG. 3 depicts components of the infusion set in a disassembled state according to one or more embodiments shown and described herein;

FIG. 4 depicts a sectional view of the infusion set according to one or more embodiments shown and described herein;

FIG. 5 depicts a diagrammatical illustration of an infusion system including the infusion set according to one or more embodiments shown and described herein;

FIG. 6 depicts a diagrammatical illustration of an infusion system according to one or more embodiments shown and described herein;

FIG. 7 depicts a diagrammatical illustration of an infusion system according to one or more embodiments shown and described herein;

FIG. 8 depicts a diagrammatical illustration of an infusion system according to one or more embodiments shown and described herein;

FIG. 9 depicts a top view of the contact sensor element or unit of a delivery supervisory installation according to one or more embodiments shown and described herein;

FIG. 10 depicts a cross-sectional view (A-A) of the contact sensor element or unit of FIG. 9 according to one or more embodiments shown and described herein;

FIG. 11 depicts a detailed view of FIG. 10 according to one or more embodiments shown and described herein;

FIG. 12 depicts a non-contact sensor of the delivery supervisory installation according to one or more embodiments shown and described herein;

FIG. 13 depicts a non-contact sensor of the delivery supervisory installation according to one or more embodiments shown and described herein;

FIG. 14 depicts a delivery supervisory installation according to one or more embodiments shown and described herein;

FIG. 15 depicts a delivery supervisory installation according to one or more embodiments shown and described herein;

FIG. 16 depicts a schematic structural view of an infusion system according to one or more embodiments shown and described herein;

FIG. 17 depicts a perspective view of an infusion system according to one or more embodiments shown and described herein; and

FIG. 18 a cross-sectional view of a subcutaneous portion of an infusion cannula comprising a subcutaneous contact sensor element or unit according to one or more embodiments shown and described herein.

### DETAILED DESCRIPTION

Embodiments of the present disclosure described herein provides the administration of medicaments with infusion systems comprising an infusion pump and an infusion set which can be worn locally independent from the infusion pump at an infusion site directly on a person's skin, the infusion set being small and inexpensive.

One embodiment described herein is directed to an infusion set for administering a medicament delivered by an infusion pump which can be carried separately from the infusion set. In operation, when administering the medicament, the infusion set is attached directly at an infusion site on a person's skin while the infusion pump can be carried locally independent from the attached infusion set above or under the clothing, for example, at a belt or in a pocket, fluidically connected with the infusion set by flexible tubing. Infusion pumps of this type are known for example, from EP 0 991 440 B1, EP 0 985 419 B1, EP 1 633 414 B1 and EP 1 716 879 B1, incorporated by reference herein.

In another embodiment, the infusion set comprises an adhesive underside for attachment at the infusion site on the skin, an infusion cannula projecting from the underside, and a first connector for fluidically connecting the cannula to the infusion pump to infuse the medicament delivered by the infusion pump. The infusion set can be fixed at the infusion site with its underside in adhesive contact with the skin surface. The infusion set can comprise a flexible pad which forms the adhesive underside. The adhesive underside can alternatively be formed, for example, directly on the underside of a relatively stiff base member of the infusion set. The cannula can be a piercing needle or a soft cannula which is placed in the body tissue with the aid of a needle which is withdrawn after placement of the cannula. The cannula is arranged such that it is placed in the body tissue automatically together with placement of the infusion set on the skin. The cannula is a subcutaneous cannula, for example, it projects over the underside of the infusion set with a length for skin penetration and subcutaneous placement of a cannula tip. In such embodiments in which the infusion set is a subcutaneous infusion set, the cannula projects from the underside with a length of about 4-12 mm.

The infusion cannula is the only skin piercing or penetrating element of the infusion set, and is a single-lumen cannula. A single-lumen cannula is advantageous as it facilitates the use of a cannula which is small in outer diameter. This helps to reduce perception of pain associated with penetration of the skin. If the cannula is placed in the body tissue with the aid of a needle, the needle is regarded as a part of the cannula, only aiding in placing the cannula in the body tissue. In operation of the infusion set, after having pierced or penetrated the skin, only the cannula is placed in the body tissue and the needle retracted or disposed.

In another embodiment, the infusion set is an infusion-only set which only serves the purpose to deliver the medicament. The infusion-only set is not equipped with means for sensing in the body tissue, in-vivo, any therapy relevant parameter characteristic for the person's health condition. A health characteristic parameter which can be used to control the feeding activity of the distant infusion pump, for example, the glucose level in the blood or in subcutaneous tissue, can be measured by means of a separate monitoring system. Separating such a monitoring function from the infusion set facilitates small size, has contact surface and low weight and price.

In another embodiment, the infusion set comprises at least one electrically powered functional component which improves medicament administration, for example, by stimulating the body tissue at the infusion site or supervising the flow of the medicament through the infusion set. To relieve the infusion pump from providing the electrical energy necessary to operate the at least one functional component, the infusion set includes its own energy source for electrically powering the functional component. The energy source can be provided, for example, as an electric battery or accumulator, a fuel cell, a photovoltaic cell, an electromagnetic generator for transforming motion energy into electrical energy or a thermo cell that transforms thermal energy from the patient's skin into electrical energy. A depleting energy source, such as a battery, accumulator or fuel cell, may be supplemented by a non-depleting, regenerative energy source, such as a photovoltaic cell, an electromagnetic generator or a thermo cell. In embodiments in which the infusion set comprises both types of energy sources the power management can be designed such that the functional component is powered by the depleting energy source during times where the regenerative energy source cannot generate electrical energy or only in insufficient amounts, and the regenerative energy source powers the functional component or supplements the depleting energy source during all other times. If the infusion set is equipped with further functional components requiring electric power, each further component is also electrically powered by on-board energy sources. An external energy source for electrical energy is not required to operate the single or plural electrically powered functional components. The infusion set is autonomous with respect to electric energy required for its own integrated operational functionality.

In another embodiment, the infusion set is subdivided into a disposable part and a reusable part. The disposable part comprises at least one component or structure of the infusion set which requires short term replacement, and the reusable part comprises at least one component of longer operational lifetime. The disposable part can comprise structures or components which come into contact with body tissue. The disposable part forms the underside for attachment on the skin and comprises the infusion cannula and also the first connector for the fluidic connection with the infusion pump. The reusable part comprises at least one energy source and a housing, for example, a cap, for protectively accommodating the energy source. The disposable part further comprises a second connector and the reusable part further includes a third connector. The second and third connectors mating one with the other to directly interconnect the disposable part and the reusable part such that, both parts form a compact unit in the interconnected state.

The reusable part and the energy source or sources may constitute a unit which is replaceable only by a new reusable unit. The reusable part may alternatively accommodate the energy source such that the energy source, once depleted, can be replaced by a new one and the remainder of the reusable part be used together with the new energy source.

In a further embodiment, the infusion set improves therapy and the administration of the medicament by means of the at least one electrically powered functional component. The improvement may reside in affecting the body tissue at the infusion site in stimulating the body tissue, or in affecting or being affected by the medicament flow, in supervising the medicament flow through the infusion set, or in providing an alert to indicate that replacement of the disposable or reusable part, of the functional component or the energy source is needed, or some malfunction has been detected. Size and weight are kept low due to the restriction of the infusion set to medicament delivery, to infusion only.

In another embodiment, the infusion set comprises at least one functional component, a stimulator, a delivery supervisory installation or a lifetimer.

In another embodiments, the infusion set includes an alarm means, for example, a buzzer, or a manipulator for manual manipulation of the infusion set.

A stimulator is a heating or vibratory stimulator for mechanically stimulating the tissue at the infusion site. The stimulator is designed such that it affects the outer skin surface thereby stimulating the subcutaneous tissue. If the stimulator is a heating stimulator it can be an ohmic resistor. The stimulator is optionally part of the underside of the disposable part covered with adhesive material. A heating stimulator surrounds the infusion cannula at least partially or all around. The stimulator's length and width, seen in a view onto the underside, is larger than its thickness. The stimulator may be a mechanically stimulator or a vibratory stimulator located at the underside of the disposable part and at least partially surrounds the cannula. The stimulator can also be a heating and vibratory stimulator in combination, for example, an ohmic resistor which is set in vibratory motion while heating at the same time or heating and vibrating, one operation timely separated from the other.

The at least one functional component can either be disposed at the disposable part or at the reusable part or can comprise a component part disposed at the disposable part and a further component part disposed at the reusable part. The expression “disposed at” comprises arrangements on an outer surface of the respective carrier part, namely the disposable or reusable part, as well as arrangements in which the respective component or component part is disposed in a recess or cavity of or is completely enclosed, for example, embedded in the respective carrier part. It defines, however, that the respective component or component part is a member only of that one of the two carrier parts at which it is disposed, once the two carrier parts have been disconnected one from the other. The explanation of “disposed at” holds not only with respect to the at least one functional component powered by the energy source but also with respect to any other kind of means, for example, the energy source disposed at either the disposable or the reusable part. Should the at least one functional component be disposed at the disposable part, the disposable and the reusable part, and thereby the electrically powered functional component and the energy source, can electrically be connected by means of a galvanic contact or by induction wherein the electric connection is a direct connection between the disposable and the reusable part. A galvanic contact is can for example be formed as a male and female connection, a plug and socket connection. A galvanic connection can alternatively be formed as a pressure contact, the pressure being exerted by an elastic resilience of either only a connecting means of the disposable part or the reusable part or an elastic resilience of the connecting means of both parts, wherein the pressure exerted in the contacted state is advantageously parallel to a direction into which the reusable part is attached to the disposable part to establish the interconnected state of these parts. The electric connection is established in all embodiments requiring for the transmission of electrical power automatically by interconnecting the disposable and the reusable part. An additional manual action is advantageously not required. The connecting means for the mechanical interconnection and the connecting means for the electric connection are in all embodiments designed and arranged such that both kinds of connections are established by the same relative movement the two module parts have to accomplish for being interconnected.

In one embodiment, the infusion set comprises a delivery supervisory installation having at least one of a plurality of functional components. The delivery supervisory installation supervises the delivery of the medicament by sensing a characteristic variable of the medicament delivery or more specifically of the medicament flow, for example, the rate of flow, possible air bubbles in the flow or the fluid pressure. The delivery characteristic variable, or the flow characteristic variable can be sensed within or at the infusion cannula. The variable can instead or in addition be sensed upstream of the cannula and downstream of or directly at the first connector, the fluidic connector of the infusion set. The delivery characteristic variable can be sensed with the aid of a sensor which is in contact with or disposed in a medicament feeding line extending from an upstream end of the first connector to the cannula tip. Such the sensor is a contact sensor element or unit. The contact sensor element or unit is in direct contact with the feeding line or the medicament optionally disposed on an outer circumferential surface of the feeding line. The contact sensor element or unit can alternatively be embedded in the feeding line or attached to the inner surface of the feeding line or, as a further alternative, be an integrated part of the feeding line.

In one embodiment, the delivery supervisory installation supervises the flow of the medicament between the upstream end of the first connector and the upstream end of the cannula. In another embodiment the delivery supervisory installation is located between the upstream end of the cannula and the cannula tip.

In a further embodiment, supervision of a delivery characteristic variable can be accomplished by means of a contact sensor element or unit which experiences a deformation, optionally an elastic deformation, in dependence on the fluid pressure in the feeding line. The deformation or degree of deformation is detected by a further sensor means, without direct contact, for example, by a non-contact sensor. The detection can be an optical one. The contact sensor element or unit is disposed at the disposable part and the non-contact sensor disposed at the reusable part. Detection is accordingly performed via outer surfaces of the two module parts, these surfaces facing each other and being internal surfaces of the infusion set in the interconnected state of the disposable and the reusable part. A sensor means of the disposable part, for example, the contact sensor element or unit, can be passive, for example, does not need to be powered. An alternative contact sensor element or unit may be, for example, a strain gauge which, if disposed at the disposable part requiring a power transmission via an electric interface between the disposable and the reusable part.

In another embodiment, the contact sensor element or unit can comprise a micro-fluidic chamber having a bottom substrate and a top cover, the top cover being spaced from the bottom substrate so as to define a height (H1) of the chamber. One or more walls or fillings can be positioned in the chamber, the walls or fillings defining a fluid channel there between such that the fluid channel extends from an inlet of the chamber to an outlet of the chamber. Each of the walls or fillings has a height (H2) less than the height (H1) of the chamber so as to define a fluid gap between a top surface of each wall or filling and the top cover. The dimensions (H1, H2) of the walls or fillings and the chamber are chosen such that the fluid gap will be filled with liquid by capillary forces via the fluid channel when liquid is introduced into the fluid chamber. The fluid gap adjacent to a section of the fluid channel filled by a liquid introduced into the fluid chamber will be filled with said liquid by capillary force. The top cover is a flexible, resilient membrane deforming in dependence on the medicament fluid pressure.

In another embodiment, the delivery supervisory installation is a pressure sensor comprising the micro-fluidic chamber and a detecting means arranged to measure the deformation of the top cover of the micro-fluidic chamber. At least a part of the surface of the bottom structure or the walls or the top cover facing toward an inner volume of the chamber can be hydrophilic. The fluid channel has a meander-like shape. The non-contact sensor is arranged to measure a deformation of the top cover of the micro-fluidic chamber. Similarly, a micro-fluidic chamber can aid in degassing the medicament fluid thereby serving as a degasser. The top cover can be gas-permeable for degassing. In a modification, the chamber is of dimensions above the micro range so capillary forces do not play a role or decisive role. A meander-like shape of the feeding line increases the flow resistance in the region of the sensor membrane.

In another embodiment, the contact sensor element or unit operates as a pressure sensor comprises a stack of layers, optionally coplanar layers, with a rigid top layer and a rigid base layer, a resilient metallic electrode layer and a metallic counter-electrode layer, the electrode layer and the counter-electrode layer being electrodes of a sensing capacitor, and a spacer layer which has a through cut-out, the through cut-out defining an electrode cavity, wherein the fluid channel is coupled to the electrode layer such that a fluidic positive pressure of the drug in the fluid channel causes the electrode layer to bend into the electrode cavity, thus modifying the capacitance of the sensing capacitor. The contact sensor element or unit of this type constitutes a delivery supervising sensor on its own. The contact sensor element or unit is expediently disposed at the disposable part and connected electrically with the energy source via an electrical connection of the two module parts.

In a further embodiment, at least two electrodes can be provided. Whereby at least one of the electrodes can be a subcutaneous electrode comprised by the infusion cannula. The other of the at least two electrodes can also be disposed at the cannula, as a subcutaneous electrode. The at least one other electrode can instead be disposed in the feeding line upstream of the cannula either still at the infusion set or at the infusion pump or in the tubing for fluidically connecting the infusion set with the infusion pump. An electrode disposed in the tubing or at the pump can be operated with successive subcutaneous electrodes which get disposed with the disposable part of the infusion set. An impedance measuring means is operatively coupled to the at least two electrodes and is further designed to measure at least one impedance value between the at least two electrodes. An event trigger means is operatively coupled to the impedance measuring means and is designed to evaluate the at least one impedance value and to generate an event trigger if evaluation of the at least one impedance value indicates the occurrence of a delivery anomaly. At least one of the impedance measuring means and the event trigger means can be disposed at the reusable part of the infusion set.

As used herein, the following terms have the following meanings unless expressly stated to the contrary:

The term “impedance” is used in the sense of an electrical impedance which may comprise ohmic, as well as capacitive or inductive components. The term “impedance value” refers to a complex value or a vector of values reflecting either or all of the impedance components. In some embodiments described herein, the impedance is an ohmic resistance and the corresponding impedance value is a resistor value. However, capacitive or inductive impedance components may be evaluated alternatively or additionally to an ohmic impedance component. The term “impedance value” may further be referred to as a value correlated with and derivable from an impedance or impedance component, such as a specific conductivity, capacity, or the like as well as to an electrical measuring value correlated with an impedance or impedance component, such as the voltage drop over an impedance. The impedance is especially defined by the subcutaneous tissue or the drug which is administered by the infusion device.

The term “subcutaneous electrode” refers to an electrode which is placed in the subcutaneous tissue. Such a subcutaneous electrode may be arranged inside the infusion cannula or at the outer surface of the infusion cannula in an area which is placed in the subcutaneous tissue during application.

The at least one subcutaneous electrode and the at least one further electrode are completely or partly coated by substantially inert layers of gold, silver, platinum, or the like or may be made from substantially inherent materials.

The contact sensor for impedance measurement is best understood based on a consideration of the subcutaneous tissue impedance, or the specific ohmic resistance of the subcutaneous tissue and of the medicament fluid. The specific ohmic resistance of the subcutaneous tissue and thus ohmic resistance which may be measured between two electrodes placed in the subcutaneous tissue is under normal conditions largely given by the specific ohmic resistance of the interstitial fluid. The specific ohmic conductivity, for example, the reciprocal of the specific ohmic resistance, of the interstitial fluid is about 15.8 mS/cm. The specific ohmic conductivity of insulin formulations and a number of further liquid medicaments is about 2 mS/cm. In a stationary state with no medicament being administered or having been administered for some time, the specific impedance and the specific ohmic resistance of the subcutaneous tissue is largely determined by interstitial fluid. If medicament is administered, the interstitial fluid is, at least partly, temporarily displaced by medicament fluid in an area around the administration aperture at the distal tip of the infusion cannula, the medicament forming a subcutaneous medicament depot. Accordingly, the specific impedance and the specific ohmic resistance of the subcutaneous tissue around the infusion cannula shows a temporary variation, the temporary variation especially involving a temporary increase and a peak in the specific ohmic resistance. After completing the administration, the specific impedance returns to its initial value along with the medicament being absorbed by the subcutaneous tissue. Variation of the specific impedance with respect to time is reflected by a corresponding variation of a impedance value measured between two subcutaneous electrodes which are located at fixed positions in the subcutaneous tissue.

In addition, the medicament concentration during and immediately after administration decreases with increasing distance from the administration aperture. During and immediately after administration, the specific impedance of the subcutaneous tissue is therefore non-uniform with respect to position. The specific ohmic resistance decreases with increasing distance from the administration aperture. Accordingly, the specific ohmic resistance or any component of the tissue impedance may therefore be considered as scalar field having an specific impedance gradient. In the case of no impedance gradient, that is, in case of a uniform spatial impedance distribution, the impedance value measured between two subcutaneous electrodes is substantially proportional to the distance between the electrodes. This is not the case if an impedance gradient is present, that is, for a non-uniform spatial impedance distribution. The specific impedance variation along a given axis may therefore be determined by measuring impedance values between at least three electrodes of given distances placed along the given axis. Variation of the specific impedance along the axis is reflected by the impedance values measured between pairs of the at least three electrodes not being proportional to the distance between the electrodes. The axis may be the cannula axis. For simplicity reasons, the term “impedance distribution” is used for the spatial distribution of the specific impedance of the subcutaneous tissue.

The variability of the specific impedance with respect to time or position may be evaluated for administration supervision and administration anomaly detection. If, for any reason such as an occlusion, a leakage, a disconnected infusion cannula, a device fault, or the like, the medicament is not administered, this variability will not occur. In at least one embodiment described herein, the impedance measuring means is designed to monitor the at least one impedance value as a function of time or position, and the event trigger means is designed to generate an event trigger if the at least one impedance value as a function of time or position indicates the occurrence of an administration anomaly.

In another embodiment, the infusion set can be equipped with a lifetimer, in addition to one or more of the other functional components. The lifetimer can be designed such that it alerts the person at expiration of a predetermined time of use of the disposable part, for example, to remind the person that the disposable part needs regular replacement. Similarly, the lifetimer may be designed to alert the person that the energy source is depleted or will reach a predetermined end of operational lifetime requiring for replacement only of the energy source or of the disposable part as a whole. The alert is an acoustic or vibratory alert or both either in combination or sequentially one after the other, for example, in alternation. If a vibratory stimulator is present, the alert should be distinct from the vibratory stimulating operation of such a stimulator. The lifetimer includes a clock which is started either manually by actuating an optional actuator at the time the disposable part is attached on the skin or automatically by establishing the interconnection of the disposable part and the reusable part or by establishing the interconnection of the disposable part and the tubing.

In another embodiment, the infusion set comprises a manipulator to actuate the at least one or at least one of a plurality of on-board functional components. The manipulator can be a key or button, for example, a turn-button, a push-button, a squeeze element or some other type of actuator for manual activation. A manipulator can be disposed at the infusion set alternatively or in addition to a remote manipulator, for example, of the infusion pump. The manipulator may be disposed at the reusable part.

In a further embodiment, the functional component is a motion sensor detecting and recording motional activity of the user. A motion sensor can, for example, be formed by an acceleration sensor. The motion sensor may serve the purpose to assess the patient's physical activity, for example, for record keeping purposes or for controlling the administration. For example, a physical activity may be detected and, depending thereon, a temporary reduction of a basal administration be recommended to the patient or automatically carried out. With a motion sensor it can, for example, be detected if the patient is awake or asleep. The detection of exceptional events, for example, a mechanical shock might be entered to the device history. The lifetimer or the motion sensor, if present, may be disposed at the reusable part.

In another embodiment, the functional component is a temperature sensor, for example, to sense ambient temperature and thereby detect possible exposition to exceptionally high or low temperature that can be entered into a storage means for the device history. A temperature sensor can additionally or alternatively be disposed such that correct attachment of the infusion set to the skin can be supervised by means of such a temperature sensor which can be disposed at the underside of the disposable part to contact the skin or to be in close vicinity of the skin. A temperature sensor can alternatively serve the purpose to supervise a stimulator for stimulating the body tissue by heating.

In another embodiment, the functional component is a skin contact sensor which, besides the temperature sensor as mentioned above, may be a skin impedance sensor or an electrical switch contact that is closed or opened by the skin contact.

In another embodiment, the functional component is a humidity or liquid sensor for detecting medicine leakage or for supervising correct operation of electrical contacts, if the disposable part comprises electrically powered components.

In a further embodiment, the functional component is a perspiration sensor sensing and recording perspiration directly at the contact interface between the underside of the infusion set and the skin. A perspiration sensor can, for example, comprise electrodes located at the underside of the disposable part measuring the electrical conductivity on the surface of the skin.

In another embodiment, the infusion set comprises an alarm means for releasing an acoustic or vibratory alarm. The alarm means can be part of or coupled to a stimulator or a delivery supervisory installation or a lifetimer or some other kind of functional component. The alarm means can also be coupled to and shared by two or more different functional components of the infusion set. The alarm means, if present, may be disposed at the reusable part.

In another embodiments the infusion set comprises a signal processing or control unit which can be designed either only for on-board processing of signals from an on-board functional component formed as a sensor means or only for controlling the operation of an on-board functional component, for example, the stimulator or a sensor means. The signal processing or control unit is a signal processing and control unit for both signal processing and controlling. The processing or control unit is disposed at the reusable part. The signal processing function can, for example, reside in transforming analogue signals of a sensor means in digital data which can be transmitted to the pump. The signal processing or control unit can include a data storage means for storing digital data. The signal processing or control unit can also include a processing unit, for example, for recognition of hazardous situations sensed with the aid of a delivery supervisory installation. Such a processing capability would relieve the infusion pump if alarms are to be created by the pump and would simplify integration of the infusion set in the control system of the pump.

In a further embodiment, the infusion set comprises sensor means, for example, a delivery supervisory installation, used to amplify the outgoing sensor signals on-board the infusion set. The sensor means is disposed at the disposable part, a signal amplifier can be disposed at the disposable part to transmit amplified signals to the infusion pump or to the signal processing or control unit.

In another embodiment, the disposable part delivers the medicament at the infusion site without the reusable part being interconnected. Feeding of the medicament is via the disposable part. The reusable part is fluidically isolated from the disposable part and has no physical contact with the medicament. The disposable part can be used without the reusable part. This applies irrespective of the kinds of functional components the infusion set is equipped with for improving therapy, for example, for enhancing the reliability that the medicament dose is correctly delivered or for improving reception of the medicament in the tissue or alerting the person that the disposable part has reached its end of operational life for which it was designed.

In a further embodiment, the disposable part delivers the medicament at the infusion site fluidically bypassing the reusable part or without the energy source of the reusable part or any electrical or optical coupling with the reusable part or even without the reusable part being interconnected not only for isolating the reusable part fluidically from the disposable part but also, for example, for using the infusion set or only the disposable part in combination with an infusion pump which has not the capability to operate in adapted combination with the one or more functional components electrically powered by the energy source of the reusable part or does not support the one or more functional components or is even incompatible to said one or more functional components of the infusion set. For such infusion pumps the reusable part can even be put aside by the user or can be omitted, for example, the infusion set be delivered with only the disposable part. Producing infusion sets with the disposable and the reusable part and also infusion sets with only the disposable part can furthermore reduce the production costs because of production scale effects, for example, higher production numbers of the disposable parts. Under these circumstances two different infusion sets are available, namely a first infusion set comprising the disposable and the reusable part of the present disclosure and a further infusion set. This further infusion set can consist solely of the disposable part as disclosed or can comprise such a disposable part and a further part designed to be substitutional for the reusable part of the present disclosure. This further part may serve simply as a cover for the disposable part. In such embodiments this further part is expediently designed to be disposable together with the disposable part of the present disclosure. The further part can comprise connecting means similar or identical to that of the reusable part of the disclosure for a mechanical interconnection with the disposable part of the present disclosure. This further part may serve as a covering part which covers in the interconnected state the upper side of the disposable part of the present disclosure. This covering part can advantageously provide for a smooth upper surface of the further infusion set.

In another embodiment, the disposable part and the reusable part can be interconnected mechanically if the disposable part does not comprise a functional component which needs electric energy. In another embodiment, the two parts are interconnected not only mechanically but also electrically by direct galvanic contact or a direct contactless interface for transmittance of energy to operate an active functional component of the disposable part. An optical interconnection can comprise one or more optical fibers interconnected physically, or it can be some other type of an optical interface allowing optical beams to pass through, for example, a window or breakthrough. The interconnection can alternatively be only a mechanical and optical one, for example, as described above in connection with another embodiment of a delivery supervisory installation. In further embodiment, the disposable part is interconnected with the reusable part mechanically, electrically and optically.

In another embodiment, the infusion set comprises a transmitter for transmitting signals to the infusion pump, for example, an alarm signal if an alarm means is not present at the infusion set or for creating an alarm in addition at the infusion pump. The transmitter may alternatively or in addition to transmitting an alarm signal be capable of transmitting sensor signals or data, for example, signals or data of a delivery supervisory installation or some other type of sensor like the mentioned motion sensor or perspiration sensor.

In another embodiment, the infusion set may comprise a receiver, either alternatively or in addition to a transmitter, for receiving signals from the infusion pump or an optional extra monitoring system for monitoring a therapy relevant health condition of the person, for example, by monitoring the glucose level. A receiver can, for example, be coupled with the stimulator, if present, such that the infusion pump or the extra monitoring system can automatically or the person can manually activate the stimulator at the infusion pump or with the aid of some other remote control unit. A delivery supervisory installation or some other sensor means on-board the infusion set might also or alternatively be designed for a remote activation via the on-board receiver.

The transmitter or receiver or an on-board transceiver is designed for wireless communication, for example, via infrared (IR) or radio-frequency (RF) like Bluetooth™. Signal transmission, if present, can alternatively or in addition be accomplished by means of the flexible tubing which connects the infusion set and the infusion pump fluidically in use of the infusion system. Wired signal or data transmission can be performed optically, for example, via optical fibres on or embedded in the flexible tubing. Such a solution requires optical coupling means of the infusion pump as well as of the infusion set.

In another embodiment, the infusion set may comprise flexible tubing for connecting the infusion set fluidically with the infusion pump. The tubing can be connected directly with the disposable part. The tubing can comprise an upstream connector for connecting the tubing releasably with the infusion pump and a downstream connector for connecting it releasably with the infusion set. In alternative embodiments also including flexible tubing for the interconnection with the infusion pump such tubing can be connected, unreleasably, with the infusion set and comprise a connector for a releasable connection with the infusion pump only at its upstream end. In other embodiments the infusion set can furthermore comprise an ampoule or some other type of reservoir prefilled with a medicament. In such embodiments, the ampoule or other type of reservoir will or can be disposed together with the disposable part of the infusion set. The tubing can, in such embodiments, be connected unreleasably either with the infusion set or the ampoule or other type of reservoir, or both, or can alternatively be connected via respective connectors releasably with the infusion set and also releasably with the ampoule or other type of disposable reservoir.

In a further embodiment, an infusion set which is modular in that a disposable part is combined with a reusable part and comprises at least one of the functional components, the stimulator or the delivery supervisory installation, without the on-board energy source. Such a modified modular is electrically powered by an external energy source, for example, an energy source disposed at the infusion pump and electrically connected with the at least one electrically powered functional component via the fluidic tubing. The energy source can in principal be disposed at some other device external to the infusion set, for example, at an external energy module for electrically powering only the infusion set, for example, the at least one functional component, or for electrically powering the infusion set and the pump. Therefore, the embodiment disclosed herein, and not limited thereto is a modular infusion set on its own and in combination with an external infusion pump regardless of where the energy source is disposed.

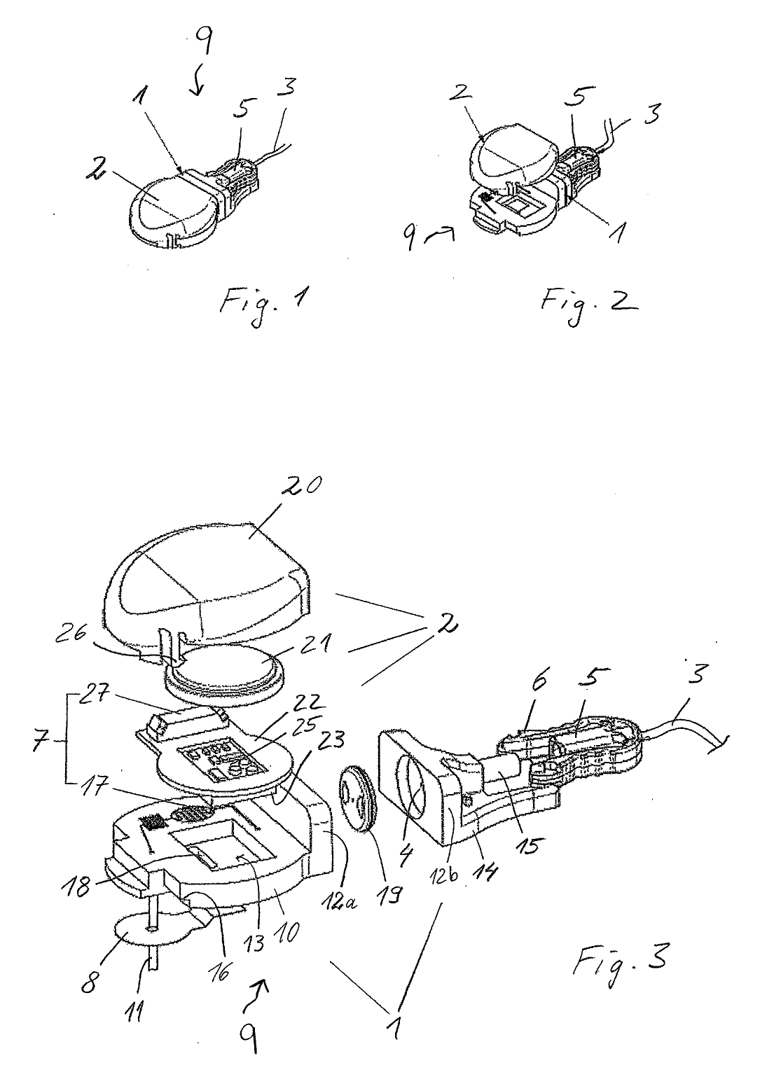

Referring now to the drawings, FIG. 1 shows an infusion set which can be attached on the skin of a person to administer a medicament fluid into the body tissue at the infusion site through an infusion cannula which projects from the underside of the infusion set. The infusion set is generally indicated in FIG. 1 as reference symbol 9. The underside of the infusion set 9 is covered with an adhesive to fix the infusion set on the skin. The infusion set 9 is modular in that it comprises a disposable part 1 forming the underside which is the skin contact surface of the infusion set, and a reusable part 2 which is releasably interconnected with the disposable part 1. The infusion set 9 is shown in FIG. 1 with the two module parts, the disposable part 1 and the reusable part 2 in the interconnected state. The interconnection is a direct interconnection, for example, the two parts, the disposable part 1 and the reusuable part 2 are mechanically interconnected, one mechanically directly with the other. The infusion set 9 is fluidically connectable to an infusion pump by means of flexible tubing 3. In operation, when medicament is infused, a feeding means of the infusion pump feeds the medicament from a reservoir of the infusion pump via the tubing 3 to the infusion set 9 and via the cannula into the body tissue. The cannula can be a subcutaneous cannula projecting from the underside of the infusion set with a length suitable for subcutaneous delivery of the medicament. The flexible tubing 3 is fluidically and mechanically connected to the infusion set 9 by means of a fluidic connector 5. The connector 5 constitutes the downstream end of the tubing 3. A similar connector may constitute the upstream end of the tubing 3 to releasably connect the tubing 3 with the infusion pump which may be worn remote from the infusion set 9. In another embodiment, the downstream connector 5 of the tubing 3 may be omitted and the tubing 3 be unreleasably connected with the infusion set 9. In such embodiments, the upstream connector of the tubing 3 can constitute the first connector of the infusion set 9. In still further embodiments, the tubing 3 can be connected unreleasably with the infusion set 9 and furthermore be connected directly with an ampoule prefilled with the medicament or some other type of reservoir prefilled with the medicament such that the ampoule or other type of reservoir is disposed off, after use, together with the disposable part 2. In such embodiments, the ampoule or other type of reservoir can constitute the first connector of the infusion set, however, more preferred, the tubing 3 is provided with a downstream connector, for example, the connector 5 for a releasable connection with the infusion set 9. In all embodiments in which the tubing 3 comprises a downstream connector for fluidically connecting the tubing 3 with the infusion set 9 this connector and a counter connector of the infusion set 9 may be designed such that a tubing 3 which is provided separately from the infusion set 9 or as a separate part of the infusion set 9 can be connected with the disposable part 2 only once. In such embodiments, the mechanical interconnection once established is unreleasable in order to prevent that the tubing 3 can be reused without the disposable part 2.

FIG. 2 shows the infusion set 9 with the reusable part 2 disconnected from the disposable part 1. The disposable part 1 is connected with the tubing 3 by means of the connector 5. Medicament can be infused while the reusable part 2 is disconnected since the medicament flow is through the tubing 3 and only the disposable part 1. The reusable part 2 is fluidically isolated from the flow of the medicament.

The two module parts, the disposable part 1 and the reusuable part 2, as seen in FIG. 1, are forming a low profile, for example, a flat infusion set 9 in their interconnected state. The reusable part 2 is located in the interconnected state on top of the disposable part 1 to reduce the area of the infusion set 9 when seen in a top view onto the upper side of the infusion set 9. The total area covered by the infusion set 9, in the skin attached state is roughly the same as that of the disposable part 1 alone.

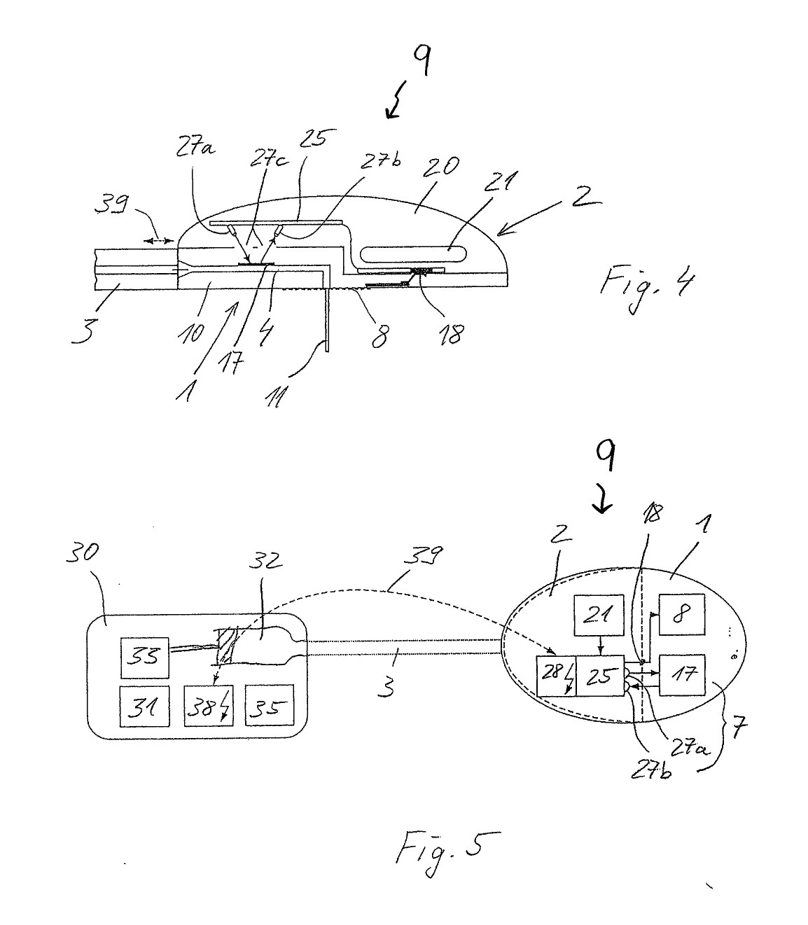

In FIG. 3 the infusion set 9 is illustrated in a disassembled state of components. The disposable part 1 constitutes a basic structure of the infusion set and comprises a primary base member 10 and a secondary base member 14 which are formed separately and jointly fixed one to the other to form the unitary base members 10, 14. The base members 10 and 14 form a flat base comprising the adhesive underside for skin attachment and an upper side opposite to the underside. Base member 14 is formed with a first connector 15, a fluid connector, for releasably connecting the connector 5 to the disposable part 1 such that medicament can flow via the fluidic connection of the interconnected connectors 5 and 15 into a feeding line 4 in which the medicament is fed via the disposable part 1 to an upstream end of the cannula 11. The feeding line 4 and the cannula 11 can be formed as a unitary part that is embedded in the base member 10 and leaves the base member 10 via a curvature at the underside to constitute the cannula 11. The upstream end of the cannula 11 is located at the underside of the disposable part 1, flush with the underside. Regardless of how the feeding line 4 and the cannula 11 are formed, when speaking of the cannula 11 only the projecting length is meant.

Base member 10 comprises an upright structure 12a projecting from the upper side of base member 10, and base member 14 comprises an upright structure 12b projecting from the upper side of base member 14. In the assembled state the base members 10 and 14 are joint together with their upright structures 12a and 12b being fixed one to the other. The fluid connector 15 projects from the upright structure 12b in a small distance from the upper side of the base member 14 and parallel to the underside of the disposable part 1 such that connector 5 can be interconnected with connector 15 in a relative motion parallel to the underside of the disposable part 1. Connector 5 comprises locking means 6 which automatically interconnect mechanically with counter locking means of the disposable part 1, with counter locking means of the base member 14. The base members 10 and 14 can in alternative embodiments be formed in a single piece, however, forming the base members 10, 14 in two or even more different pieces facilitates the forming process and offers the opportunity to dispose an air degasser 19 in the medicament flow. Air degasser 19 is accommodated in a chamber which is jointly formed by the base members 10 and 14.

In another embodiment, the infusion set 9 comprises at least one electrically powered functional component to improve the fluid flow function and reception of the medicament at the infusion site. For example, one of the functional components may be a delivery supervisory installation 7 for supervising the flow and the delivery of the medicament. In another example, one of the functional components may be a stimulator 8 for stimulating the body tissue at the infusion site to improve the reception of the medicament in the body tissue. The stimulator 8 is a heating means for stimulating the body tissue by heating the skin in direct heat contact. The delivery supervisory installation 7 is a flow detector for supervising the flow of the medicament through the feeding line 4.

The stimulator 8 is an electric (ohmic) resistor disposed at the underside of the disposable part 1 in direct vicinity of the cannula 11. The cannula 11 projects through a passage formed in the stimulator 8 which closely and completely surrounds the cannula 11 directly at the underside of the disposable part 1. The stimulator 8 is a flat plate-like structure or a foil structure.

The stimulator 8 is electrically powered by an energy source 21, an electrical battery or accumulator, which is an internal on-board energy source of the infusion set 9. The energy source 21 is a flat and disc-like body. The energy source 21 is accommodated in a chamber of the reusable part 2, the chamber being formed by a cap 20 and a base member 22 of the reusable part 2. Cap 20 forms a top surface of the reusable part 2 and infusion set 9 oppositely facing away from the underside of the disposable part 1. Base member 22 forms an underside of the reusable part 2, this underside being in direct contact with the upper side of a flat base portion of the base members 10, 14 of the disposable part 1 when the disposable and reusable parts 9 are in the interconnected state.

The reusable part 2 furthermore comprises an electronic control unit 25 controlling the stimulating action, or heating action of the stimulator 8. The stimulator 8 is electrically connected to the control unit 25 via an electric connection means 18 of the disposable part 1 and is electrically connected to the energy source 21 via the control unit 25. Control unit 25 is disposed at the reusable part 2 and disposed at the base member 22.

The disposable part 1 and the reusable part 2 are mechanically directly connected one to the other by means of a releasable interlocking action of mechanical connectors 16 of the disposable part 1 and mechanical counter connectors 26 of the reusable part 2. The connectors 26 are formed as hooks which grip behind the connectors 16 when the reusable part 2 is brought at its underside in contact with the upperside of the flat base portion of the disposable part 1. The energy source 21 is galvanically connected with the stimulator 8 automatically at the same time the mechanical interconnection of the disposable and reusable parts 1 and 2 is established.

The delivery supervisory installation 7 comprises a contact sensor element or unit 17 disposed at the disposable part 1 and a non-contact sensor 27 disposed at the reusable part 2. The non-contact sensor 27 is accommodated in the chamber formed by the base member 22 and cap 20 of the reusable part 2. Contact sensor element or unit 17 is disposed to be in optical contact with the non-contact sensor 27 when the two module disposable and reusuable parts 1 and 2 are interconnected. Contact sensor element or unit 17 comprises or consists of an elastic membrane which is deformed in dependence on the fluid pressure in the feeding line 4. The non-contact sensor 27 detects the state of deformation of the contact sensor element or unit 17 and thus flow of the medicament through the feeding line 4. The contact sensor element or unit 17 is passive, for example, does not require power supply. Non-contact sensor 27 is the active part of delivery supervisory installation 7 and is electrically powered by the energy source 21, for example, completely internal of the reusable part 2 and powered via the control unit 25.

The control unit 25 controls the operation of delivery supervisory installation 7 and also the operation of the stimulator 8. The control unit 25 is also a signal processing means processing the output signals received from the non-contact sensor 27. This processing can comprise transforming analogue output signals of the non-contact sensor 27 into digital data. The processing capability can include outputting data to the infusion pump. The signal processing function of the control unit 25 can be restricted to outputting analogue sensor signals to the infusion pump, however transforming these signals into digital data on-board the infusion set and outputting the data or only part of the data. The control unit 25 can include a data storage means for storing of the sensor data or only of selected data, for example, event data, after further processing. Event data can, for example, be data representing the exceedance of a predetermined threshold level of, for example, the fluid pressure.

The electrical energy required for these operations and also the operation of the control unit 25 is provided alone by the internal energy source 21. The infusion set 9 is electrically self sustaining, no electrical energy from outside, for example, the infusion pump. The modular infusion set 9, namely the energy source 21, the control unit 25 and the non-contact sensor 27 are integral to the reusable part 2, whereas fluid flow communication between the down-stream end of tubing 3 and the cannula 11 is assigned completely to the disposable part 1. Therefore, the disposable part 1 can be used without the reusable part 2.

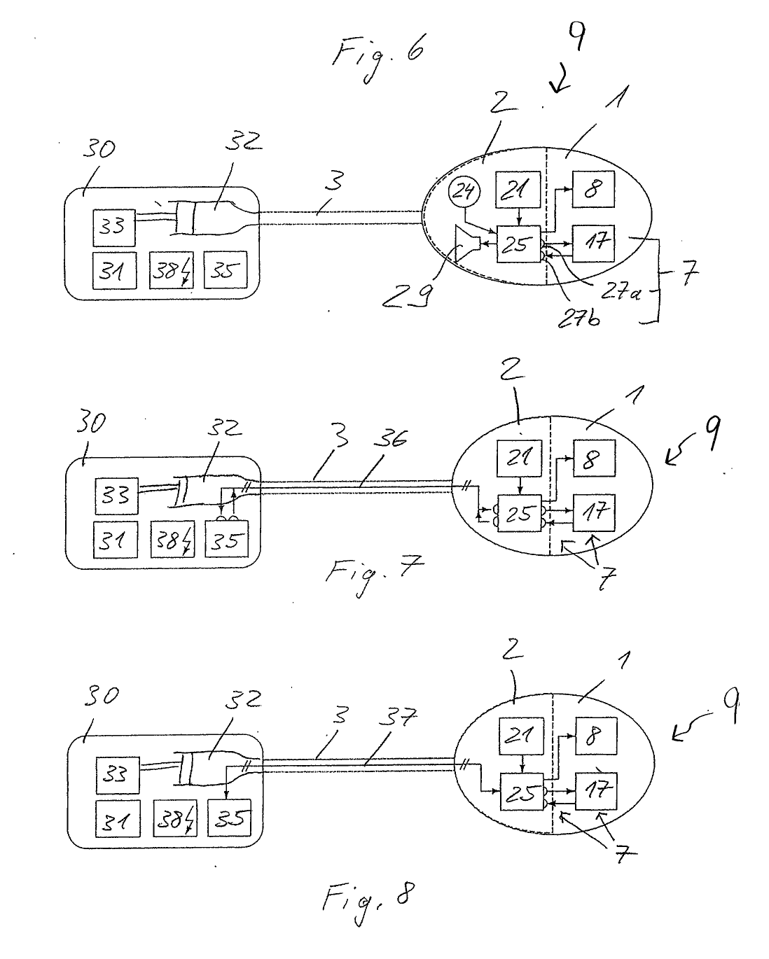

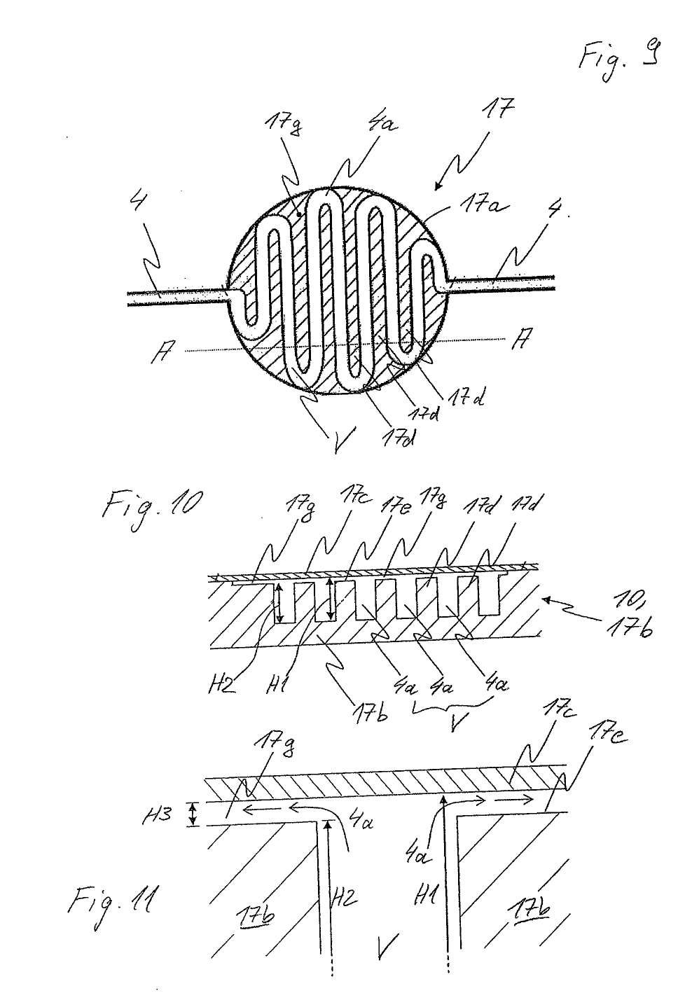

FIG. 4 shows the infusion set 9 in a section along the feeding line 4. The stimulator 8 is accommodated in a flat recess at the underside of the disposable part 1 to form the underside as a continuously smooth contact surface. The electrical connecting means 18 is resiliently urged against a contact means of the reusable part 2 to galvanically connect the stimulator 8 via the control unit 25 to the energy source 21. The contact sensor element or unit 17 is attached to the outer circumferential surface of the feeding line 4. The contact sensor element or unit 17 may alternatively form part of the feeding line 4. The contact sensor element or unit 17 is elastically deformed in dependence on the fluid pressure within the feeding line 4. The non-contact sensor 27 is of the optical type. The non-contact sensor 27 comprises an optical emitter 27a, for example a light emitting diode (LED), which directs an optical beam onto the contact sensor element or unit 17, and an optical receiver 27b which receives the reflected beam as an optical signal. The optical beams are transmitted via the contact surfaces of the disposable and reusable parts 1 and 2, for example, via the underside of the reusable part 2 and the upper side of the disposable part 1 which together form the optical interface 27c by direct contact. The optical elements, emitter 27a and receiver 27b, are electrically powered by the energy source 21 and operationally controlled by the control unit 25.

The disposable part 1 and the reusable part 2 are interconnected mechanically and in addition coupled electrically and optically. In embodiments not comprising energy consuming components like a stimulator 8 powered by the on-board energy source only the optical coupling is provided by or via the optical interface 27c could be present. In other embodiments, without optical interface, for example, without any optical component but with a powered component, only the electrical coupling could be present. The mechanical interconnection however remains in both embodiments.

FIG. 5 is a diagrammatical illustration of an infusion system including an infusion pump 30 which can be worn, for example, under the clothing on a belt or in a pocket. The infusion set 9 can be attached at the infusion site on the skin independently of the location of the infusion pump 30. The fluidic connection is established, as explained in connection with FIGS. 1 to 4, by means of flexible tubing 3. The infusion pump 30 comprises a reservoir 32 containing the medicament, for example, an ampoule; a feeding means 33 for feeding the medicament from the reservoir 32 via the tubing 3 and through the infusion set 9 into the body tissue. Feeding means 33 is powered by an energy source 31 on-board the infusion pump 30. In alternative embodiments not shown, the energy source 31 can be arranged external of the infusion pump 30, for example, as an integral part of a remote control unit connected to the infusion pump 30 by wire or wireless for wired or wireless power supply or wired or wireless signal communication. Such a combination of a light-weight infusion pump and remote control unit is disclosed in EP 1 633 414 incorporated by reference herein. Feeding means 33 is controlled by an electronic control unit 35 on-board the infusion pump 30. Parts of the control unit 35 may alternatively be external as disclosed in EP 1 633 414.

The infusion pump 30 and the infusion set 9 are coupled by wireless communication 39. The wireless communication 39 is bidirectional. The infusion set 9 comprises an on-board transceiver 28 communicating, in the coupled state, bidirectionally with a transceiver 38 on-board the infusion pump 30. The wireless communication 39 may be unidirectional, the infusion set 9 either only receiving control signals from or only transmitting sensor signals or data or other information or alarm signals to the infusion pump 30. The wireless communication 39 can, for example, be a RF communication, a Bluetooth™ communication. If the infusion pump 30 can transmit control signals to the infusion set 9, via either a bidirectional or unidirectional wireless communication 39, the control signals can be signals to activate the stimulator 8, optionally to deactivate the stimulator 8, or to activate the delivery supervisory installation 7, optionally to deactivate the delivery supervisory installation 7. If the infusion set 9 is transmitting signals or data to the infusion pump 30 via the bidirectional wireless communication 39 or instead by a only unidirectional wireless communication 39, such signals or data can be sensor signals or data of the delivery supervisory installation 7, for example, to inform the user of a malfunction or of proper flow of the medicament. The signals or data may be recorded in a memory of the infusion pump 30.

FIG. 6 is a diagrammatical illustration of the infusion system in accordance with the various embodiments of the present disclosure. The infusion system comprises a infusion pump 30 and an infusion set 9 which can be attached on the skin locally independent of the infusion pump 30. The infusion set 9 differs from that of the other embodiments disclosed herein in that the infusion set is not provided with the capability of signal or data transmission to or from the infusion pump 30. There is neither a transmitter nor a receiver disposed at the infusion set 9 as disclosed in other embodiments. The infusion set 9 is only fluidically connected to the infusion pump 30.

In a further embodiment, the infusion set 9 comprises a manipulator 24 for manually activating the stimulator 8. The on-board manipulator 24 replaces the remote actuation by means of the infusion pump 30.

In another embodiment, the infusion set 9 comprises an acoustic alarm means 29. The acoustic alarm means 29 can optionally be replaced by a vibratory alarm means or by an acoustic and vibratory alarm means. The manipulator 24 and the alarm means 29 are coupled with the electronic control unit 25. The stimulator 8 is activated by actuating the manipulator 24 which can be provided, for example, as a pushbutton. Actuation of the manipulator 24 activates the stimulator 8 and, at the same time, a time lapse system, for example, a clock, also disposed at the infusion set 9 as an integral part of the control unit 25. At expiration of a certain time limit, for example, 5 to 20 seconds, predetermined by the time lapse system the alarm means 29 releases an acoustical alarm. The person perceiving the alarm signal will then actuate the infusion pump 30, directly or by means of a remote control unit if such is present, and the infusion pump 30 will deliver a bolus of medicament. The infusion pump 30 is equipped with a transmitter or receiver or a transceiver 38, however, the transceiver 38 does not communicate with the infusion set 9. It is provided for communication with, for example, a remote control unit for remotely controlling the infusion pump 30, if applicable, or with a separate health parameter monitoring system, if the infusion system 9, 30 is part of an infusion and monitoring system.

The manipulator 24 can alternatively be coupled to the delivery supervisory installation 7, via the control unit 25. In such embodiments, an actuation of the manipulator 24 will either activate the delivery supervisory installation 7, or both, the stimulator 8 and the delivery supervisory installation 7.

According to a further embodiment, but not limited thereto, the infusion set 9 includes an on-board manipulator 24 or an on-board alarm means 29, one or both of these components being provided in addition to the remote communication capability of the infusion set 9.

FIG. 7 is a diagrammatical illustration of the infusion system in accordance with the various embodiments of the present disclosure. The infusion set 9 is coupled to the infusion pump 30 via the tubing 3 not only fluidically but also optically for signal or data transmission which may be a bidirectional signal or data transmission. The tubing 3 is provided with a communication link 36, for example, optical fibres, connecting the infusion set 9 to the infusion pump 30 via respective optical couplings at the ends of the tubing 3. The control unit 25 is optically coupled with the control unit 35 of the infusion pump 30.

FIG. 8 is a diagrammatical illustration of the infusion system in accordance with the various embodiments of the present disclosure wherein the tubing 3 provides for the fluidic connection and also for signal or data transmission between the infusion pump 30 and the infusion set 9. The signal or data transmission is accomplished by a galvanic communication link 37 provided as a part of the tubing 3.

The infusion sets 9 of the various embodiments disclosed in FIGS. 6 to 8 comply with the infusion set 9 of the embodiment disclosed in FIG. 5 in all respects not explicitly mentioned above.

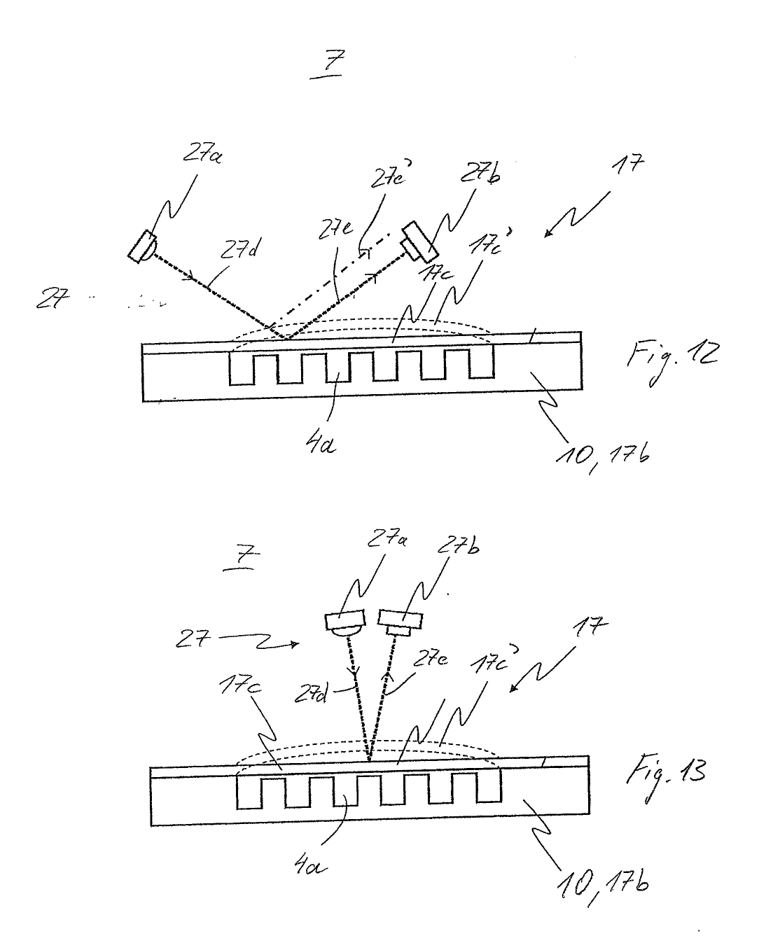

FIGS. 9 to 11 show various embodiments of the contact sensor element or unit 17. Contact sensor element or unit 17 comprises a micro-fluidic chamber 17a. The oval, in top view, for example, circular micro-fluidic chamber 17a comprises a bottom substrate 17b formed in the base portion of the base members 10, 14 of the disposable part 1 and a top cover 17c. The top cover 17c is spaced from the bottom substrate 17b by a certain height (H1), thus defining an inner volume (V) of the micro-fluidic chamber 17a. Eight walls 17d are arranged in the micro-fluidic chamber 17a, and define a meander-like fluid channel 4a that runs from an inlet to an outlet of feeding line 4. The first connector 15 and the cannula 11 are connected by the fluid channel 4a which forms a section of the feeding line 4.

The height (H2) of the walls 17d is less that the overall height (H1) of the micro-fluidic chamber 17a. As a result there is a fluid gap 17g between the top cover 17c and the upper surface 17e of the walls 17d, with a height (H3=H1−H2). The dimensions of the micro-fluidic chamber 17a and the walls 17d, particularly the heights (H1), (H2), (H3) are chosen such that there are non-negligible capillary forces acting on a fluid present in the micro-fluidic chamber 17a. Fluid in the fluid channel 4a will be dragged by said capillary forces into the fluid gap 17g.

The specific dimensions depend on the liquid used, and on the properties the upper surfaces 17e of the top cover 17c and the top of the walls 17d, since this will eventually define the interface tensions between liquid, surfaces, and gas/air in the micro-fluidic chamber 17a, which then will define the effective capillary forces for a certain geometric setting of the micro-fluidic chamber 17a. Since in most cases liquid medicaments are aqueous solutions, it is preferable that at least the most relevant surfaces, namely the upper surface 17e of the walls 17d and the surface of the top cover 17c facing toward upper surface 17e are hydrophilic, with a contact angle <90°, in order to increase the overall capillary effect. For aqueous liquids a preferred range for the height H3 of the fluid gap 17g lies between 20 and 200 μm, and preferably between 50 and 150 μm.

The dimensions of the micro-fluidic chamber 17a and the fluid channel 4a are less critical. A typical diameter of a micro-fluidic chamber 17a may, for example, lie between about 2 to 10 mm. The fluid channel 4a may have a width of, for example, 0.1 to 1 mm, while the height (H2) of the walls 17d lies in a range between 0.25 to 5 mm, or optionally lies between 0.5 and 1 mm. The aspect ratio between the width of the fluid channel 4a and the height (H2) can lie between 0.25 and 5, and is preferably about 1.

When the micro-fluidic chamber 17a is filled through the inlet with a liquid, the liquid will flow essentially along the fluid channel 4a. The capillary forces will drag liquid in the fluid channel 4a into the adjacent sections of the fluid gap 17g, effectively supplanting air present in the gap. It is energetically much more favorable for air to form spherical bubbles with minimum surface toward the hydrophilic surroundings, and thus no air bubbles remaining in the fluid gap 17g.

The capabilities of the micro-fluidic chamber 17a are independent from its orientation in space. Since the capillary forces and interface tensions responsible for the smooth filling of the gap are much stronger than the gravitational force acting on the liquid, and the buoyancy force acting on the air bubbles in the liquid, the micro-fluidic chamber will be completely filled with liquid independent on its orientation. Thus, the filling behavior of such a micro-fluidic chamber 17a, are predictable and reproducible.

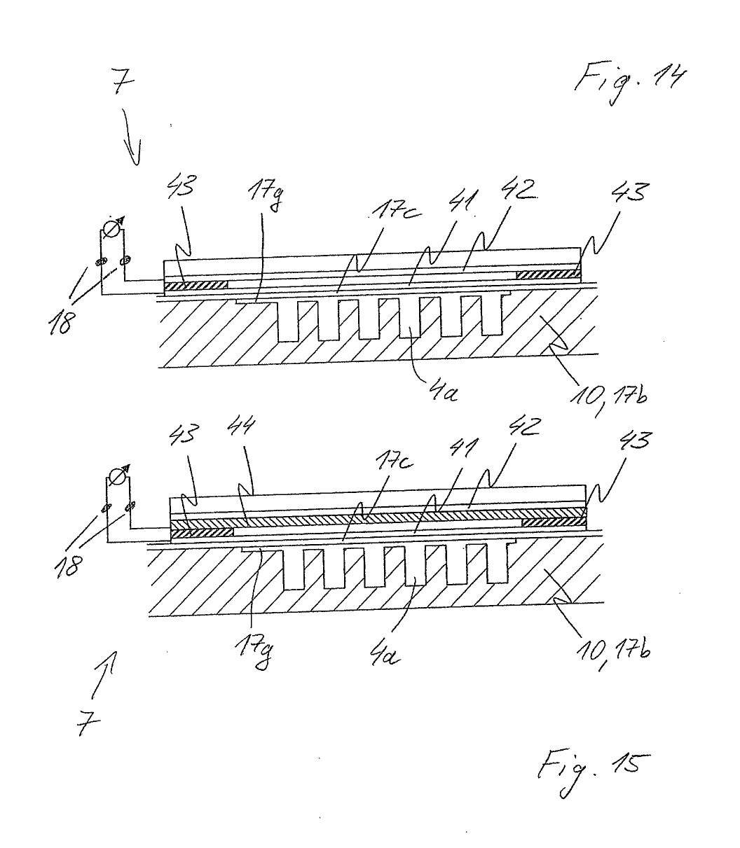

Other embodiments of the delivery supervisory installation 7 including the contact sensor element or unit 17 of FIGS. 9 to 11 are shown in FIGS. 12 and 13, both based on optical principles. The top cover 17c is a flexible, resilient membrane sealed to the base member 10 along the outer rim of the micro-fluidic chamber 17a. The optical emitter 27a, such as, for example a light emitting diode (LED) or a laser diode, and the optical receiver 27b, such as, for example, a photo diode or a photo transistor, are arranged such that an incident light beam 27d emitted by the optical emitter 27a is reflected by the surface of the top cover 17c toward the optical receiver 27b, where it is detected. The top cover 17c may be metal vapor coated to increase reflection. A metal coating for increasing reflection may also be realized by galvanic or chemical deposition or by a sandwich structure of a metal and a non-metal layer. When the top cover 17c bulges under a positive pressure difference (dashed lines 17c′) the reflected light beam 27e does not impinge any longer on the optical receiver 27b. In such an embodiment the detection system thus delivers a binary on/off signal correlated to a certain pressure threshold, which can be used by a control unit of the distant infusion pump. Such a system detects occlusion in a fluid line. To achieve a higher resolution in the pressure values, a sensor receiver array can be used instead of a single sensor optical receiver 27b. The pressure values are used by the control unit 25 or a control unit of the pump to calculate the current flow of liquid and the administered dose of liquid medicament or for detecting a steady pressure increase over time as indication for an occluded cannula.

In a further embodiment, the optical delivery supervisory installation 7, shown in FIG. 13, where the optical emitter 27a and the optical receiver 27b are arranged in such a way that the reflected light beam 27e will fall onto the optical receiver 27b independent of a displacement of the top cover 17c. The position of the surface of the top cover 17c is determined by analyzing the amplitude of the reflected light, which depends on the length of the combined light path of the incident light beam 27d and the reflected light beam 27e.

In further embodiments, the non-contact sensor 27 can comprise two or more of the optical receivers 27b. The optical receivers 27b are arranged such that the reflected light beam 27e will always fall onto at least one of the optical receivers 27b at any state of deformation of the top cover 17c. A non-contact sensor 27 modified this way guarantees that a sensor output signal is created at any pressure level in the feeding line 4, or to be more precise in the fluid channel 4a.

In the embodiments with an optical supervisor installation 7, for example, the embodiments according to FIGS. 9 to 13, the disposable part 1 and the reusable part 2 are coupled optically via the respective optical interface 27c which is simply a break-through provided by respective openings at the mating surfaces of the disposable and reusable parts 1 and 2.

In another embodiment the delivery supervisory installation 7 operates as a pressure sensor with a contact sensor element or unit 17, for example, the micro-fluidic chamber 17a with a top cover 17c formed as an elastic membrane, as illustrated in FIGS. 14 and 15. The displacement of the flexible membrane, top cover 17c is determined by measuring a capacitance. The delivery supervisory installation 7 is disposed at the disposable part 1 and electrically connected via electric connection means 18 with the reusable part 2 firstly, for electrically powering the contact sensor element or unit 17 and secondly, for transmitting the sensor output signals via the electric connecting means 18 to a capacitance measuring means disposed at the reusable part 2. The electric connection means 18 is the interface for the transmission of both, electric energy to the contact sensor element or unit 17 and sensor output signals to the signal processing and control unit of the infusion set, optionally formed by a control unit 25 according to the embodiments described in FIGS. 1 to 8 but adapted to the delivery supervisory installation 7 of the capacitance type, which modified control unit includes a capacitance measuring means. Wherein a first capacitor electrode 41, for example, a thin metal foil, is arranged adjacent to the flexible top cover 17c membrane. Whereas, the flexible top cover membrane 17c can be realized as a first capacitor electrode 41, for example, by coating it with a conducting material.

In another embodiment as shown in FIG. 14, insulating spacer elements 43 define a distance between said first capacitor electrode 41 and a second capacitor electrode 42, located on top of the spacer elements 43 and the first capacitor electrode 41. The first and second capacitor electrodes 41 and 42 are electrically isolated from each other, and thus act as a capacitor with a capacitance (C), which can be measured. With increasing internal pressure in the micro-fluidic chamber 17a, the flexible, resilient membrane top cover 17c bulges outwards. The first capacitor electrode 41 is displaced towards the second capacitor electrode 42. As a result the capacitance (C) increases, which can be detected and used to determine the deformation of the top cover 17c and the internal pressure in the micro-fluidic chamber 17a causing said deformation, respectively.

When the internal pressure is high enough the first capacitor electrode 41 will eventually touch the second capacitor electrode 42, and the ohmic resistance (R) between the two layers drops to zero. This event can also be detected by suitable electronic means, for example, the on-board control unit 25, and can be used in addition or as an alternative to the capacitance as an input for a control system of the infusion system.

In another embodiment, the capacitive delivery supervisory installation 7 is shown in FIG. 15, where an additional insulating layer 44 is arranged between the spacer elements 43 and the second capacitor electrode 42. Said additional insulating layer 44 inhibits a short-circuit between the first and second capacitor electrodes 41 and 42, depending on the used capacitance measurement circuitry.

In another embodiment, the second capacitor electrode 42 can be located on the opposite side of the micro-fluidic chamber 17a, below the bottom substrate 17b, or integrated into the bottom substrate 17b.

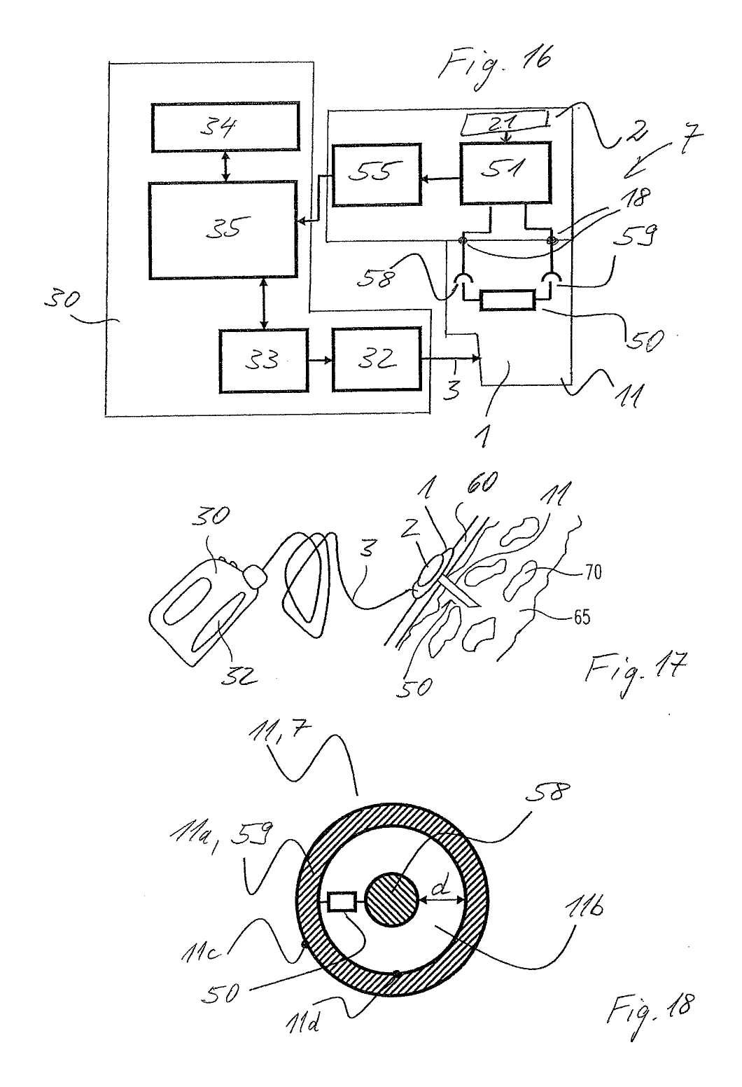

FIGS. 16 to 18 show a further embodiments of the infusion system. The infusion system comprises an infusion pump 30 with a medicament reservoir 32, feeding means 33, control unit 35 and a user interface 34, the user interface 34 being designed to indicate alarms. The infusion system furthermore comprises an infusion set with a disposable part 1 and a reusable part 2. The disposable part 1 comprises an infusion cannula 11 with the distal cannula tip. An impedance measuring means 51 and an event trigger means 55 are disposed at the reusable part 2. The infusion cannula 11 is made of medical grade stainless steel and is fluidically connected with the reservoir 32 via the infusion tubing 3. The cannula 11 further comprises two subcutaneous electrodes 58 and 59 as electrodes which are coupled via a coupling impedance 50 having an impedance value (R). The coupling impedance 50 is not an electric component but is given by the medicament or subcutaneous tissue coupling the electrodes. The electrodes 58 and 59 are operatively coupled to the impedance measuring means 51. The electrodes 58 and 59 are connected with the on-board energy source 21 as described in at least one of the embodiments shown in FIGS. 3 to 8. The electrodes 58 and 59 form the contact sensor element or unit of the delivery supervisory installation 7.

The impedance measuring means 51 is designed to measure an ohmic resistance as impedance value (R) and may be of any kind known in the art. The impedance measuring means 51 is operatively coupled to the event trigger means 55 which is designed to evaluate the impedance value (R) and generate an event trigger. Either or both of the impedance measuring means 51 or the event trigger means 55 may be, fully or partly, integral with a control unit like the control unit 25 described in at least one of the embodiments shown in FIGS. 1 to 8 or be disposed as additional equipment.

Upon reception of an event trigger generated by the event trigger means 55, the control unit 25 shown in FIGS. 3 to 8, generates an alert or error message or a warning which is indicated to the user via the user interface 34 and stops further medicament administration. The user interface 34 of the infusion pump 30 comprises optical indicators, such as a display as well as acoustical and/or tactile indicators, such as a buzzer and/or a pager vibrator.

In a further embodiment, the components of the infusion pump 30 are enclosed by a common device housing shown in FIG. 17. Alternatively, the infusion pump 30 may be split into two or more separate units which are physically or operatively coupled. For example, the user interface 34 may be made by a remote controller, a cell phone, or the like, and may communicate with the control unit 35 via a wireless data interface.

The infusion cannula 11 penetrates the skin 60 in a substantially perpendicular manner and is placed in the subcutaneous tissue 65, the subcutaneous tissue 65 having interstitial fluid 70.

FIG. 18 shows a cross sectional view of the infusion cannula 11, substantially being a cylindrical tube having a cannula wall 11a and an administration aperture 11b at its distal tip. The electrode 58 is a subcutaneous center electrode and is arranged in the center of the administration aperture 11b. The second electrode 59 is a subcutaneous counter electrode which is made by the cannula wall 11a. The subcutaneous center electrode 58 and the subcutaneous counter electrode 59 have a radial distance (d) which may be in a range of, for example, 0.05 mm to 0.5 mm. Alternatively, the cannula 11 may be made from a non-conductive material, such as Teflon. In this embodiment, the subcutaneous counter electrode 59 may be designed as a ring or ring segment and arranged at the cannula circumferential outer or inner surface 11c or 11d.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modifications and variations come within the scope of the appended claims and their equivalents.

What is claimed is:

1. An infusion set for administering a medicament delivered by an infusion pump which can be carried separately from the infusion set, the infusion set comprising:
a disposable part; a reusable part; and an electrically powered functional component,
wherein the disposable part comprises:
an adhesive underside for attachment at an infusion site on a person's skin,
a single lumen infusion cannula which projects from the underside and is the only skin piercing or penetrating element of the infusion set,
a first connector which fluidically connects the cannula to the infusion pump, and
a second connector, and
wherein the reusable part comprises:
an energy source which electrically powers the functional component, and
a third connector mated with the second connector of the disposable part to interconnect the disposable part and the reusable part,
wherein the reusable part is in the interconnected state fluidically isolated from the disposable part, and
wherein the disposable part comprises a feeding line fluidically connecting an upstream end of the first connector with a downstream end of the cannula to feed and deliver the medicament via the disposable part and bypass the reusable part.

2. The infusion set of claim 1, wherein the functional component is at least one of a stimulator for heating or mechanically stimulating the tissue at the infusion site and a delivery supervisory installation for supervising the delivery of the medicament by sensing a variable which is characteristic for the medicament flow downstream of or at the first connector.

3. The infusion set of claim 2, wherein the stimulator forms part of the underside of the disposable part at least partially surrounding the cannula.

4. The infusion set of claim 2, wherein the delivery supervisory installation comprises a contact sensor element or unit disposed at the disposable part and a non-contact sensor disposed at the reusable part.

5. The infusion set of claim 4, wherein the contact sensor element or unit is in contact with the feeding line for the medicament or directly with the medicament, the feeding line fluidically connecting an upstream end of the first connector with a downstream end of the cannula, and the contact sensor element or unit and further comprises a deformation structure which deforms in dependence of a fluid pressure within the feeding line.

6. The infusion set of claim 4, wherein the non-contact sensor provides for contactless supervision of medicament flow through the feeding line of the infusion set, the feeding line fluidically connecting an upstream end of the first connector with a downstream end of the cannula.

7. The infusion set of claim 4, wherein the non-contact sensor is arranged for sensing the contact sensor element or unit optically.

8. The infusion set according to claim 1, wherein the reusable part further comprises a manipulator to provide manual manipulation of the stimulator.

9. The infusion set according to claim 2, wherein the functional component further comprises:
a lifetimer, disposed at the reusable part, which alerts the person at expiration of a predetermined time of use of the disposable part; and
an alarm means, disposed at the reusable part, which releases an acoustic or vibratory signal.

10. The infusion set according to claim 9, wherein the functional component is at least the stimulator and further comprises:
a motion sensor, disposed at the reusable part, which senses motional activity of a person;
a perspiration sensor in contact with the underside of the disposable part which senses perspiration at the surface of the skin;
a temperature sensor, disposed at the underside of the disposable part, which senses ambient or skin temperature and supervises the stimulator; and
a skin contact sensor which senses whether the infusion set is in contact with the skin.

11. The infusion set according to claim 1, wherein the reusable part further comprises a transceiver electrically powered by an energy source to transmit signals to or receive signals from the infusion pump via a wireless communication for infusion-set-internal signal communication.

12. The infusion set of claim 1, wherein the reusable part comprises a signal processing or control unit for controlling the functional component or processing signals received from the functional component, and the energy source electrically powers the signal processing or control unit.

13. The infusion set of claim 1, wherein the infusion set is electrically or optically coupable or coupled with the infusion pump for signal or data transmission via a fluidic tubing; and the infusion set comprises flexible tubing for connecting the infusion set fluidically and releasably with the infusion pump.

14. The infusion set of claim 1, wherein the reusable part is interconnected mechanically with the disposable part.

15. The infusion set of claim 1, wherein the reusable part is interconnected with the disposable part and only mechanically and electrically, only mechanically and optically, or only mechanically, electrically and optically.

16. The infusion set according to claim 1, wherein the disposable part further comprises base members which forms the underside and an upper side opposite to the underside of the disposable part, wherein the first connector protrudes from an upright structure which protrudes from the upper side and extends at least parallel to the upper side, and the reusable part is attached to the upper side of the disposable part.

17. An infusion system, comprising:
an infusion set according to claim 1;
an infusion pump with a medicament reservoir, feeding means for feeding medicament from the reservoir, and an energy source for powering the feeding means; and
flexible tubing connecting the infusion pump with the infusion set to administer the medicament from the reservoir via the cannula of the infusion set, the tubing being directly connected releasably or unreleasably with the disposable part.

18. The infusion system according to claim 17 further comprising a monitoring system which includes a sensor which senses a therapy relevant health parameter of the person, the sensor being separate from the infusion set.

19. The infusion system according to claim 18 wherein the sensor is a glucose sensor.

20. The infusion system according to claim 18 wherein the sensor senses the therapy relevant health parameter of the person in vivo.
